(19) United States
(12) Patent Application Publication
(10) Pub. No.: US 2011/0281279 A1
(43) Pub. Date: Nov. 17, 2011

(54) CIRCULATING Epha2 RECEPTOR

(75) Inventors: Peter Hamer, Reading, MA (US); Stephen P. Bradley, Brighton, MA (US); Walter Carney, North Andover, MA (US)

(73) Assignee: Siemens Healthcare Diagnostics Inc., Tarrytown, NY (US)

(21) Appl. No.: 12/808,470

(22) PCT Filed: Dec. 17, 2008
(86) PCT No.: PCT/US08/87128

§ 371 Date: Jun. 16, 2010

Related U.S. Application Data
(60) Provisional application No. 61/015,881, filed on Dec. 21, 2007.

(51) Int. Cl. G01N 33/573; C07K 16/40; G01N 33/82; G01N 21/75; C12Q 1/48; G01N 21/64

(52) U.S. Cl. 435/7.4; 435/15; 530/388.22; 530/389.1

(57) ABSTRACT

Provided are compositions, methods, and kits relating to the detection of a circulating or soluble form of the EphA2 receptor tyrosine kinase.

### FIELD OF THE INVENTION

Provided are compositions, methods, and kits relating to the detection of a circulating or soluble form of the EphA2 receptor tyrosine kinase.

### BACKGROUND OF THE INVENTION

EphA2 receptor is a 130 kDa receptor tyrosine kinase that is expressed at relatively low levels on a variety of adult epithelial cells. Kinch M S & Carles-Kinch K. Clinical & Experimental Metastasis 20:59-68, 2003. EphA2 receptor was identified in a screen of tyrosine kinases that are expressed in epithelial cells, and is one member of the larger family of Eph receptor kinases, all of which bind ligands (known as ephrins) that are anchored to the membrane of adjacent cells. Id. EphA2 receptor is strongly expressed in embryonic stem cells and during organogenesis (Lickliter J D, Smith F M, Olsson J E et al. PNAS1996; 93: 145-50), although in adults, EphA2 receptor expression appears to be largely restricted to proliferating epithelial cells, with low levels of expression occurring in adult ovary, colon, kidney, and lung epithelial cells. Lindberg R A, Hunter T. Mol Cell Biol 1990; 10: 6316-24.

EphA2 receptor is generally expressed at low levels in normal adult tissue, but is upregulated in malignant cancerous cells. Overexpression of EphA2 receptor is prevalent in a wide variety of solid tumors, including melanomas, kidney, breast, esophageal, prostate, pancreas, colon, ovarian, and lung carcinomas. For example, while not found in normal melanocytes or benign compound nevi, EphA2 receptor is highly overexpressed in malignant melanoma cells. See, e.g., Easty D J, Bennett D C. Melanoma Res 2000; 10: 401-11; Easty D J, Guthrie B A, Maung K et al. Cancer Res 1995; 55: 2528-32; Easty D J, Herlyn M, Bennett D C. Int J Cancer 1995; 60: 129-36. Expression of EphA2 receptor in resected renal cell carcinoma tumors appears to serve as a correlate for disease grade and a prognostic indicator of disease-free interval and overall survival in patients that were treated with surgery. It has been proposed that patients with renal cell carcinoma lesions exhibiting the highest expression levels of EphA2 receptor protein seem more likely to experience disease recurrence within one year after surgery and to survive for shorter periods of time. Herrem C J et al. Clinical Cancer Res 11:226-231 (2005). With regard to breast cancer, EphA2 receptor overexpression accurately predicts metastatic progression in vivo. Zelinski D P, Zantek N D, Stewart J C et al. Cancer Res 2001; 61: 2301-6; Zantek N D, Walker-Daniels J, Stewart J C et al. Clin Cancer Res 2001; 7: 3640-8. It has additionally been shown that EphA2 receptor overexpression correlates with poor prognosis in esophageal squamous cell carcinoma. Miyazaki T et al. Int J Cancer 2003; 103:657-63. The EphA2 receptor is also a marker of lung cancer recurrence and survival. See Kinch M S, Moore M-B, Harpole Jr. D H. Clin. Cancer Res. 9:613-618 (2003). In pancreatic adenocarcinoma, EphA2 receptor is a determinant of malignant cellular behavior, Duxbury M S et al. Oncogene 2004; 23:1448-56. Likewise, EphA2 receptor expression is associated with aggressive features in ovarian carcinoma. Thaker P H, et al. Clin Cancer Res 2004; 10:5145-50. The role of EphA2 receptor in pathological angiogenesis has also been established. See, e.g., Lin Y G, et al., Cancer. 2007 Jan. 15; 109(4.332-40; Larsen A B, et al., Mol Cancer Res. 2007 March; 5(3):283-93; Mudali S V, et al., Clin Exp Metastasis. 2006-23(7-8):357-65.

Because of these findings and the discovery that EphA2 receptor is linked to the regulation of cellular behaviors that promote metastasis (Zelinski D P et al. Cancer Res 2001; 61: 2301-6; Carles-Kinch K et al. Cancer Res 2001; 62: 2840-7), the receptor is considered to be a promising diagnostic benchmark and target for therapeutic applications. Assessments of EphA2 receptor expression in patient samples could have broad applicability for any or all of diagnosis, monitoring, and treatment of cancer and cancer-related pathologies.

### SUMMARY OF THE INVENTION

Provided are methods comprising detecting a soluble form of EphA2 receptor in a biological fluid of a subject.

Also provided are methods for detecting or aiding the detection of a disease state in a patient comprising determining the quantity of soluble EphA2 receptor in a biological fluid of the patient, wherein an elevated quantity of soluble EphA2 receptor compared to a threshold value indicates the presence of the disease state in the patient.

There are also disclosed methods for monitoring a patient undergoing a therapy regimen comprising determining a plurality of values corresponding to the quantity of soluble EphA2 receptor in a biological fluid of the patient over time, wherein an absence of change or a decrease in the values over time indicates a therapeutic effect of the therapy regimen in the patient, and wherein an increase in the values over time indicates the absence of a therapeutic effect of the therapy regimen in the patient.

Also provided are methods for monitoring a patient in which a disease state is known to be present comprising determining the quantity of soluble EphA2 receptor in a biological fluid of said patient, thereby obtaining a sample value, and comparing said sample value with a reference value.

The present invention is also directed to purified antibodies that specifically bind to a soluble form of EphA2 receptor, as well as kits for detecting a soluble form of EphA2 receptor in a biological fluid comprising the purified antibody for use as a detector reagent; and, instructions for use comprising a standard curve for interpolating the quantity of the soluble form of EphA2 receptor in the biological fluid.

### BRIEF DESCRIPTION OF THE DRAWINGS

FIG. 1 depicts measurements of soluble EphA2 receptor in normal and pathological serum and plasma samples.

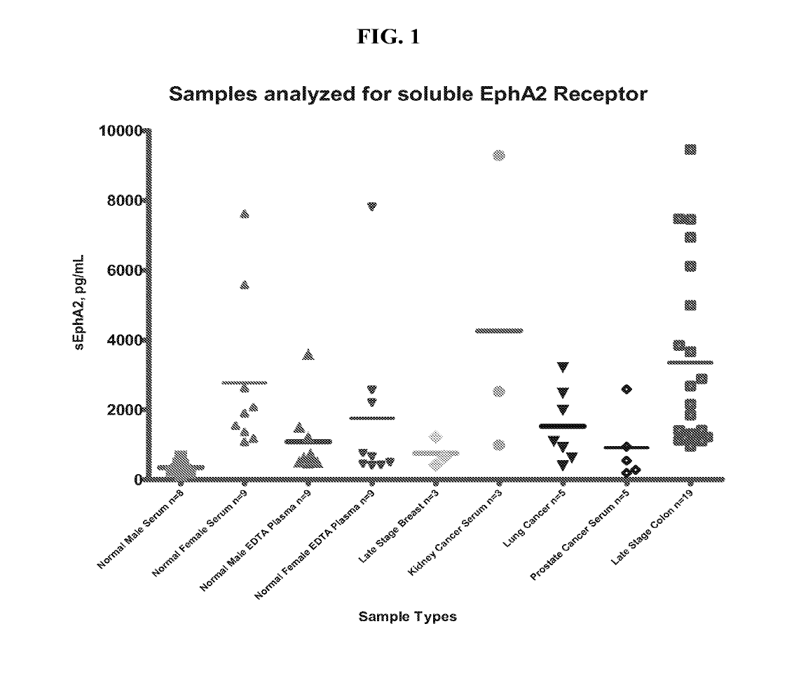

### DETAILED DESCRIPTION OF ILLUSTRATIVE EMBODIMENTS

While the Eph receptors and the ephrin family of receptors have been identified and characterized in numerous studies, circulating forms of EphA2 receptor are heretofore unknown in the literature. The present invention pertains to the novel identification of a circulating form of the EphA2 receptor tyrosine kinase, assays for the detection and quantification of circulating EphA2 receptor, and diagnostic, monitoring, and treatment methodologies that are effected through such detection, quantification, or both. As used herein, the terms “soluble” and “circulating” are interchangeable and applied in connection with the newly discovered non-membrane-bound form of EphA2 receptor.

The role of membrane-bound EphA2 receptor overexpression in the generation of metastatic phenotype has been confirmed through numerous studies, and as a result EphA2 receptor has been proposed as a promising target for diagnosis and therapeutic intervention with respect to a wide variety of carcinomas and metastatic mechanisms, such as cell growth, cell migration, and angiogenesis. See generally Kinch M S & Carles-Kinch K. Clinical & Experimental Metastasis 20:59-68, 2003, and references cited therein. See also Debinski et al., U.S. 2006/0121539 (disclosing methods for detection and diagnosis of cancer comprising detecting the only form of EphA2 receptor known at that time). Pursuant to the present invention, a naturally occurring form of soluble EphA2 receptor has been discovered for the first time. The soluble version of EphA2 receptor represents an independent prognostic and evaluative marker for carcinomas and the mechanisms of metastasis. A previous study involved the fabrication of a soluble chimeric protein consisting of the recombinant extracellular domain of the membrane-bound form of the EphA2 receptor that was fused to the human IgG1 Fc chain, and used this soluble EphA2-Fc construct to demonstrate the role of EphA2 receptor in regulating pathogenic angiogenesis, but did not identify a naturally-occurring circulating form of EphA2 receptor. See Brantley et al., Oncogene (2002) 21, 7011-7026. The utility of biomarkers to manage patient care has been firmly established, for example, in such systems as Her2/neu and Herceptin, EGFr, and IRESSA. The present discovery of naturally-occurring soluble EphA2 receptor therefore tenders new opportunities for diagnostic and therapeutic applications relating to numerous cancers and cancer-related phenotypes, among other conditions.

In the present disclosure the singular forms “a,” “an,” and “the” include the plural reference, and reference to a particular numerical value includes at least that particular value, unless the context clearly indicates otherwise. When values are expressed as approximations, by use of the antecedent “about,” it will be understood that the particular value forms another embodiment. Where present, all ranges are inclusive and combinable.

Provided are methods comprising detecting a soluble form of EphA2 receptor in a biological fluid of a subject. The phrase “soluble form of EphA2 receptor” refers to a complete molecule of EphA2 receptor tyrosine kinase that exists in circulating form, or a circulating fragment of the EphA2 receptor tyrosine kinase, such as the extracellular domain portion of the EphA2 receptor tyrosine kinase, or a fragment of the extracellular domain portion. In preferred embodiments, “soluble form of EphA2” can refer to at least the portion of the EphA2 receptor that includes one or more of the epitopes recognized by the commercially available anti-human EphA2 receptor polyclonal antibody (R&D Systems, Minneapolis, Minn.; Cat. No. 3035-A2). In accordance with the present invention, soluble EphA2 receptor may be detected in an acellular biological fluid, or in a biological fluid containing cells or cell fragments. Exemplary acellular biological fluids include, for example, acellular serum or plasma, sputum, cell-free cerebrospinal fluid, acellular saliva, sweat, tears, and urine. Whole blood, cellular fractions thereof, cell-containing saliva, tumor lysates, cerebrospinal fluid, or liquefied tissue samples are exemplary biological fluids that contain cells. Biological fluids may be obtained by conventional methods, such as biopsy to obtain tissue samples, venipuncture to obtain blood, and fractionating whole blood in order to obtain serum or plasma.

Detection of a soluble form of EphA2 receptor can be carried out using one or more assays that are known to effect the identification of proteins in a biological sample. Immunohistochemical techniques are preferred. For example, in one embodiment, a reagent comprising a monoclonal antibody that is specific to the extracellular domain of the soluble form of EphA2 receptor is incubated with the biological sample, followed by incubation with a secondary antibody that binds to the monoclonal antibody and that is conjugated to a detection moiety such as a fluorophore. Detection of the fluorophore is indicative of the presence of the soluble form of EphA2 receptor. Various acrylamide gel protein detection methods may also be used. Mass spectrometry is another widely-used protein detection method that may be used in accordance with the present invention. At least some forms of soluble EphA2 receptor may be functional, and kinase assays (i.e., measuring the phosphorylation of known EphA2 receptor substrates (see, e.g., Santoro M, et al., Endocrinology. 2004 December; 145(12):5448-51. Review) and/or binding assays for known EphA2 receptor substrates can constitute suitable means for detecting the presence of functional forms of soluble EphA2 receptor. Other assays may involve automated platforms, for example, that use magnetic particles to increase assay timing Such assays are routine and readily designed and executed by those skilled in the art.

Further to the detection of the soluble form of EphA2 receptor, the present methods may additionally comprise determining the quantity of circulating EphA2 receptor, such as to obtain a “sample value” for the biological fluid sample. As in the case of detection of a protein in a biological sample, those skilled in the art will readily appreciate that numerous techniques are available for the quantification of a protein in such sample Immunohistochemical methods, mass spectrometry, flow cytometry, kinase assays, binding assays, and gel electrophoresis, among other techniques, may suitably be used. Pursuant to the present invention, a novel enzyme-linked immunosorbent assay (“ELISA”) has been developed specifically for determining the quantity of circulating EphA2 receptor, as described in Example 1, infra.

High rates of expression of EphA2 in adult tissue are an established hallmark of cancer and related phenotypes, e.g., pathological cell growth, cell migration, and angiogenesis. Overexpression of EphA2 receptor is empirically correlated to the development of a wide variety of solid tumors, including melanomas, kidney, breast, esophageal, prostate, pancreas, colon, ovarian, and lung carcinomas. Naturally-occurring soluble EphA2 receptor represents an important new metric for the potential, presence, and/or progression, of certain disease states, most notably cancer-related disease states.

The quantity of soluble EphA2 receptor in a biological fluid of a subject can also reveal information relevant to the suitability of EphA2 receptor-targeted therapies. Previous studies have revealed that the pretreatment level of soluble biomarker can indicate whether therapies that target the cellular form of such biomarker are likely to be efficacious. For example, it is known that if pretreatment levels of circulating Her2/neu receptor, a receptor tyrosine kinase and member of the ErbB protein family, decrease within one to two weeks following Herceptin administration, then therapy that targets cellular Her2/neu receptor is not expected to produce positive results. See Köstler W J, et al., Clin Cancer Res. 2004 Mar. 1; 10(5):1618-24; Lipton A, et al., Clin Cancer Res. 2004 Mar. 1; 10(5):1559-60; Lipton A, et al., J Clin Oncol. 2003 May 15; 21(10): 1967-72; Carney W P, et al., Clin Chem. 2003 October; 49(10): 1579-98. Review; Carney, W P, Personalized Medicine 2005; 2(4):317-324. The present invention is also directed to assessing the suitability of a therapy, such as a therapy that targets cellular EphA2 receptor, based on the quantity of soluble EphA2 receptor in a biological fluid of a subject, wherein the subject has not yet undergone such therapy.

Pretreatment levels of biomarker can also correlate to patient prognosis with respect to a disease state, such as predisposition for a particular rate of progression, certain outcome, or likelihood of recurrence following treatment. Overexpression of the Her2/neu receptor in breast cancer is associated with increased disease recurrence and worse overall prognosis. Lipton A, et al., Clin Cancer Res. 2004 Mar. 1; 10(5): 1559-60; Lipton A, et al., J Clin Oncol. 2003 May 15; 21(10):1967-72; Carney W P, et al., Clin Chem. 2003 October; 49(10):1579-98. Review; Carney, W P, Personalized Medicine 2005; 2(4):317-324. The present invention may involve measuring the pretreatment quantity of soluble EphA2 receptor in a biological fluid of a subject in order to correlate such quantity to the prognosis of the subject with respect to a disease state. The prognosis of the subject may relate to the expected rate of progression of the disease state, outcome (e.g., degree of likelihood of survival), probability of recurrence following remission or treatment, or other prospective disease state parameter.

The “disease state” corresponds to any pathological condition concerning which soluble EphA2 receptor provides an indicator of presence, state of development, or both. The disease state may be a cancer or a cancer-related condition. Exemplary disease states include melanomas, kidney cancer, breast cancer, esophageal cancer, prostate cancer, pancreas cancer, colon cancer, ovarian cancer, and lung cancer, as well as pathogenic cell growth, cell migration, or angiogenesis. The disease state may be a condition that is in any stage of development or progression, e.g., an early-stage condition that has not yet symptomatically manifested itself, a partially-developed condition, a fully developed, symptomatic pathology, a pathology that is in remission, or a disease that has undergone or is undergoing any form of treatment. A condition that is in any of the traditional existential states (i.e., prodromal, incubation, icteric, or convalescence) is a “disease state” for purposes of the present invention.

It has herein been discovered that the mean value of soluble EphA2 receptor in plasma and serum of subjects in which colon cancer, renal cell carcinoma, lung cancer, and prostate cancer are known to be present is greater than the mean value of soluble EphA2 receptor in subjects in which these disease states are known to be absent, data for which is presented in Example 2, infra. It has also been discovered that the mean value of soluble EphA2 receptor in plasma and serum of subjects in which breast cancer is known to be present is less than the mean value of soluble EphA2 receptor in subjects in which breast cancer are known to be absent (see Example 2, infra). In accordance with the present invention, the “reference value” may correspond to any of a number of different parameters. For example, the reference value may correspond to the minimum quantity of soluble EphA2 receptor that is necessary to indicate the presence of a disease state. The reference value may be a single numerical value expressed in terms of, e.g., concentration (mass percentage, mass volume percentage, mass volume ratio, “parts-per” notation, molarity, and the like), with or without a standard deviation, or the reference value may comprise a range of numerical values expressed in terms of, for example, a range of concentrations, with or without standard deviations. The reference value may comprise a single data point, or may comprise an average or mean of multiple data points for the same parameter. For example, the reference value may be the quantity of soluble EphA2 receptor that was measured in a single biological fluid sample from a patient known to have breast cancer, or the reference value may be a calculated average of values obtained from a plurality (e.g., two, five, ten, twenty, one-hundred, etc.) of biological fluid samples, each from a patient known to have breast cancer. In other embodiments, the reference value corresponds to the maximum quantity of soluble EphA2 receptor that is necessary to indicate the presence of a disease state, i.e., wherein a sample value that is below such reference value is indicative of the presence of a disease state. In such embodiments, the “reference value” is defined as described above. Because it may be difficult, if not medically inappropriate, to attempt to define a single value corresponding to a quantity of soluble EphA2 receptor that represents a “tipping point” above or below which (depending on the disease state) the disease state can definitively be said to be present, a reference value that corresponds to a range of values is preferred.

The “reference value” may also relate to the quantity of soluble EphA2 receptor in a biological fluid of the same subject from which the sample value is derived, but at a different point in time. For example, the reference value may correspond to the quantity of soluble EphA2 receptor in a biological fluid of a patient at time t=1, whereas the sample value corresponds to the quantity of soluble EphA2 receptor in an aliquot biological fluid in which a measurement was performed at time t=2, where t=2 represents a later point in time than t=1. The amount of time between times t=1 and t=2 may be one or more hours, days, weeks, months, or years. Additionally, for example, time t=1 may represent a timepoint at which the subject was not undergoing a therapy, whereas time t=2 may represent a timepoint at which a therapy regimen has been commenced.

The comparison between the sample value and a reference value in accordance with the present invention can yield important information regarding the absence or presence of one or more specific disease states at a given time or over a period of time, the developmental status of one or more disease states, the prospective appropriateness of a given therapy regimen, the efficacy of an ongoing therapy regimen, and other matters of importance with regard to the treatment and/or monitoring of a subject. The instant methods may further comprise selecting a therapy regimen based on the comparison of the sample value and the reference value. The selection of a therapy regimen can embrace initiating a new therapy, altering an ongoing therapy, monitoring of a subject that is or is not undergoing therapeutic intervention, or taking no further action than had been undertaken prior to the comparison between the sample value and the reference value. For example, if the comparison between the sample value and the reference value reveals that the quantity of soluble EphA2 receptor approaches the upper end of a range of values that would be expected for a subject in which a disease state is not present, the selection of a therapy regimen may comprise initiating a protocol whereby the subject is monitored every three months, e.g., by quantitating the amount of soluble EphA2 receptor in a biological fluid of the subject once every three months and comparing the measured sample value to a reference value following each clinical visit. In another example, if the comparison between the sample value and the reference value reveals that the quantity of soluble EphA2 receptor is greater than a reference value that represents the minimum quantity of soluble EphA2 receptor that is necessary to indicate the presence of colon cancer, then such therapeutic intervention as one or more of evaluative colonoscopy, biopsy, blood counts, ultrasound, computed tomography, magnetic resonance imaging, positron emission tomography, angiography, surgery, radiation therapy, chemotherapy, and immunotherapy may be selected pursuant to developing an appropriate therapy regimen. In yet another example, if the comparison between the sample value and the reference value reveals that the quantity of soluble EphA2 receptor is less than a reference value that represents a maximum quantity of soluble EphA2 receptor that is necessary to indicate the presence of breast cancer, i.e., is less than a quantity of soluble EphA2 receptor below which there exists an indication of the presence of breast cancer, then appropriate therapeutic intervention such as mammography, biopsy, blood counts, ultrasound, computed tomography, magnetic resonance imaging, positron emission tomography, angiography, surgery, radiation therapy, chemotherapy, and immunotherapy may be selected pursuant to developing an appropriate therapy regimen.

Because patient prognosis can vary over time, it may be appropriate to perform at least one evaluation of a subject at each of multiple time points. Thus, the present methods can further comprise obtaining a plurality of sample values in a biological fluid of the subject over time. For example, one or more sample values may be obtained at timepoint t=0, one or more additional sample values may be obtained at timepoint t=1, and a subsequent sample value or set of sample values may be obtained at timepoint t=2, wherein the amount of time between t=0 and t=1 may be one or more days, weeks, months, or years, and the amount of time between t=1 and t=2 may independently be one or more days, weeks, months, or years. Sample values may be obtained at a plurality of time points that are equally spaced, or at a plurality of time points that are not equally temporally spaced. Some disease states may be associated with rapid development, and based on such factors as the type of disease state at issue and/or the degree patient predisposition for developing a disease state, it may be appropriate to obtain sample values as often as every few months, every few weeks, or every few days.

A comparison may be made between a reference value and the sample value or set of values that is obtained at each timepoint, such that a series of comparisons are performed over time. Alternately, when a plurality of sample values in a biological fluid of the subject over time, the present methods can further comprise obtaining an average of said plurality of sample values, and comparing said average with a reference value, wherein the reference value may correspond to the minimum quantity of soluble EphA2 receptor that is necessary to indicate the presence of a disease state, or wherein the reference value may correspond to the maximum quantity of soluble EphA2 receptor that is necessary to indicate the presence of a disease state, i.e., wherein a sample value that is below such reference value is indicative of the presence of a disease state.

A reference value to which the sample value is compared is preferably a “best matched” value, e.g., a sample value that was derived from serum should be compared with a reference value that was derived from serum, a sample value that was derived from plasma should be compared with a reference value that was derived from plasma, a sample value that was derived from a male subject should be compared with a reference value that was derived from a male subject, a sample value that was derived from a female subject should be compared with a reference value that was derived from a female subject, a sample value that was derived from a subject having a disease state at stage 1 should be compared with a reference value that was derived from a subject having a disease state at stage 1, and the like, depending on the type of comparison being performed.

Also provided are methods for detecting or aiding the detection of a disease state in a patient comprising determining the quantity of soluble EphA2 receptor in a biological fluid of the patient, wherein an elevated quantity of soluble EphA2 receptor compared to a threshold value indicates the presence of the disease state in the patient. The disease state may be, for example, colon cancer, renal cell carcinoma, lung cancer, prostate cancer, or pathogenic cell growth, cell migration, or angiogenesis. In other embodiments, a decreased quantity of soluble EphA2 receptor compared to the threshold value indicates the presence of a disease state in the patient. In such embodiments, the disease state may be, for example, breast cancer. The threshold value may be a single numerical value expressed in terms of, e.g., concentration (mass percentage, mass volume percentage, mass volume ratio, “parts-per” notation, molarity, and the like), with or without a standard deviation, or the threshold value may comprise a range of numerical values expressed in terms of, for example, a range of concentrations, with or without standard deviations. Because it may be difficult, if not medically inappropriate, to attempt to define a single value corresponding to a quantity of soluble EphA2 receptor that represents a “tipping point” above or below which (depending on the disease state) the disease state can definitively be said to be present, a threshold value that corresponds to a range of values is preferred.

The methods for detecting or aiding in the detection of a disease state in a patient may further comprise determining a plurality of values corresponding to the quantity of soluble EphA2 receptor in a biological fluid of said patient over time. The technique of determining a plurality of values over time may be practiced in accordance with previously described methods. It may be especially desirable to detect certain aspects of the development and progression of a disease state over time that are particularly indicative of carcinogenesis. In one embodiment of the present invention, the plurality of values corresponding to the quantity of soluble EphA2 receptor in a biological fluid of said patient over time can permit an evaluation of angiogenic progression over time. The EphA2 receptor tyrosine kinase is strongly implicated in regulating pathogenic angiogenesis (see Brantley et al., Oncogene (2002) 21, 7011-7026), and measurement of soluble EphA2 receptor over time provides valuable insights into the existence and developmental state of angiogenesis in a patient.

The instant methods for detecting or aiding in the detection of a disease state in a patient may additionally comprise selecting a therapy regimen based on the observed quantity of soluble EphA2 receptor. As provided previously, the selection of a therapy regimen can embrace initiating a new therapy, altering an ongoing therapy, monitoring of a subject that is or is not undergoing therapeutic intervention, or taking no further action than had been undertaken prior to determining the quantity of soluble EphA2 receptor.

In accordance with the present invention there are also provided methods for monitoring a patient undergoing a therapy regimen comprising determining a plurality of values corresponding to the quantity of soluble EphA2 receptor in a biological fluid of said patient over time, wherein an absence of change or a decrease in said values over time indicates a therapeutic effect of said therapy regimen in said patient, and wherein an increase in said values over time indicates the absence of a therapeutic effect of said therapy regimen in said patient. Numerous studies have confirmed the association between high rates of expression of EphA2 in adult tissue and the occurrence of cancer and related phenotypes, and an increased rate of expression of soluble EphA2 receptor over time can be strongly indicative of the development or proliferation of a disease state. An array of therapies may be available for a patient in which a disease state is known or suspected to be present, but a clinician is typically challenged to select and maintain therapeutic intervention that is suitably tailored to the needs of the patient. Especially in the case of anti-cancer therapies, treatment often involves harsh intervention, e.g., radiation therapy, chemotherapy, immunotherapy, surgery, adjuvant treatment, and neoadjuvant treatment, and it is especially critical to recommend and maintain only those therapies that are fully or partially efficacious. Thus, once a therapy regimen has commenced with respect to a patient, every effort should be made to determine the absence or presence of a therapeutic effect in such patient, and to that end the parameter of the quantity of soluble EphA2 receptor in a biological fluid of the patient over the course of the treatment, i.e., over time, can reveal whether the therapy should be maintained or discontinued. For example, if over the course of three months of treatment, the quantity of soluble EphA2 receptor in a biological fluid of said patient as measured twice per month during the three month treatment period is observed to decrease over time, a positive therapeutic effect may be attributed to such treatment. If the quantity of soluble EphA2 receptor that remains the same over time, such data may also be indicative of a therapeutic effect, as unchecked pathological progression is expected to give rise to unabated overexpression of soluble EphA2 receptor. An increase over time in the values corresponding to the quantity of soluble EphA2 receptor may indicate that the chosen therapeutic route is not fully efficacious, although a rate of increase over time that is less rapid than the rate of increase over time that was observed prior to the commencement of therapy may indicate that such therapy is partially efficacious. Thus, the instant methods may further comprise comparing the plurality of values corresponding to the quantity of soluble EphA2 receptor over time to a pre-therapy reference, wherein the pre-therapy reference corresponds to the quantity of soluble EphA2 receptor that was measured over time prior to the initiation of the therapy regimen. Preferably, the period of time over which the pre-therapy reference was obtained is substantially the same as the period of time over which the plurality of values in accordance with the present methods are determined The therapy regimen of which an evaluation is made may comprise any affirmative therapy, such as antiangiogenic therapy, radiation therapy, chemotherapy, immunotherapy, surgery, adjuvant treatment, and neoadjuvant treatment, or may comprise a combination of therapeutic approaches.

Based on the plurality of values corresponding to the quantity of soluble EphA2 receptor in the biological fluid of the patient over time, the instant methods of monitoring a patient undergoing a therapy regimen may further comprise altering the therapy regimen. Altering the therapy regimen may comprise changing the parameters of the existing therapy regimen, discontinuing the therapy regimen, incorporating one or more additional therapies, or initiating a new, different therapy. Thus, if, based on the determination of the plurality of values over time, it is deduced that an ongoing antiangiogenic therapy regimen is not efficacious, such therapy can be discontinued and possible replaced with a more aggressive treatment, such as chemotherapy or surgery.

Also provided are methods for monitoring a patient in which a disease state is known to be present comprising determining the quantity of soluble EphA2 receptor in a biological fluid of said patient, thereby obtaining a sample value, and comparing said sample value with a reference value. The reference value may correspond to the quantity of soluble EphA2 receptor that is known to be present in a biological fluid of a patient at a known stage of the disease state. Thus, for example, the reference value may correspond to the quantity of EphA2 receptor that is known to be present in a biological fluid of a patient having stage 2 colon cancer. In other embodiments, the reference value may correspond to the quantity of soluble EphA2 receptor that is known to be present in a biological fluid of said patient at an earlier time point. For example, where the sample value corresponds to the quantity of soluble EphA2 receptor in a biological fluid of the patient at time t=1, the reference value may correspond to the quantity of soluble EphA2 receptor in a different biological fluid sample of the patient, as obtained at time t=0, wherein t=1 corresponds to a time point that may be one or more hours, days, weeks, months, or years later than time t=0.

The instant methods for monitoring a patient in which a disease state is known to be present may further comprise determining a plurality of values corresponding to the quantity of soluble EphA2 receptor in a biological fluid of the patient over time. The technique of determining a plurality of values “over time” may be practiced in accordance with previously described methods. When a plurality of values over time are determined, the instant methods may additionally comprise conducting a comparison among the plurality of values, thereby determining the absence or presence of one or more trends in the quantity of soluble EphA2 receptor in the biological fluid of the patient over time. Thus, for example, the comparison among the plurality of values can be used to determine whether there exists a fully or substantially upwards trend among the plurality of values over time, a fully or substantially downwards trend among the plurality of values over time, any combination of fully or substantially upwards and fully or substantially downwards trends, or no discernable trend among the plurality of values over time.

In accordance with any of the methods of the present invention, the additional step of measuring the quantity of one or more other biomarkers may be performed. That is, the instant methods may further comprise measuring the quantity of at least one biomarker other than soluble EphA2 receptor. The additional biomarker may be one with which EphA2 receptor has a known relationship, such as VEGF/VEGFR. Previous studies have reported functional evidence for EphA class receptor regulation of VEGF-dependent tumor angiogenesis. See, e.g., Cheng N, Neoplasia. 2003 September-October; 5(5):445-56. In other embodiments, the biomarker may be one with which a formal relationship to EphA2 has not yet been established, but which is otherwise implicated in carcinogenesis, angiogenesis, cell migration, cell proliferation, or other cancer-related events. The measured quantity of additional biomarker(s) may then respectively be compared to a known reference value for such biomarker, thereby providing an additional data parameter that can be used for any purpose disclosed herein with respect to soluble EphA2 receptor. For example, with respect to the disclosed methods for monitoring a patient undergoing a therapy regimen, the methods may further comprise determining a plurality of values corresponding to the quantity of each of one or more additional markers in the biological fluid over time, thereby obtaining a value for each of said one or more additional markers for every timepoint at which a value corresponding to the quantity of soluble EphA2 receptor is determined Thus, if values corresponding to the quantity of soluble EphA2 receptor are obtained at each of time t=1 and time t=2, then a value corresponding to the quantity of each of one or more additional markers in said biological fluid may also be obtained at each of time t=1 and time t=2.

The present invention also provides a purified antibody that specifically binds to a soluble form of EphA2 receptor. The purified antibody may be monoclonal or polyclonal, the former being preferred. In one embodiment, the antibody binds to a single site on the portion of the soluble form of EphA2 receptor that corresponds to the extracellular domain of the membrane-bound form of EphA2 receptor. The extracellular domain of the membrane-bound form of EphA2 receptor is found at amino acids 25 to 534 of the EphA2 receptor tyrosine kinase, the full amino acid sequence of which is reproduced below.

```
(SEQ ID NO: 1)
                10 20 30 40 50 60
MELQAARACF ALLWGCALAA AAAAQGKEVV LLDFAAAGGE LGWLTHPYGK GWDLMQNIMN
                70 80 90 100 110 120
DMPIYMYSVC NVMSGDQDNW LRTNWVYRGE AERNNFELNF TVRDCNSFPG GASSCKETFN
              130 140 150 160 170 180
LYYAESDLDY GTNFQKRLFT KIDTIAPDEI TVSSDFEARH VKLNVEERSV GPLTRKGFYL
              190 200 210 220 230 240
AFQDIGACVA LLSVRVYYKK CPELLQGLAH FPETIAGSDA PSLATVAGTC VDHAVVPPGG
              250 260 270 280 290 300
EEPRMHCAVD GEWLVPIGQC LCQAGYEKVE DACQACSPGF FKFEASESPC LECPEHTLPS
              310 320 330 340 350 360
PEGATSCECE EGFFRAPQDP ASMPCTRPPS APHYLTAVGM GAKVELRWTP PQDSGGREDI
              370 380 390 400 410 420
VYSVTCEQCW PESGECGPCE ASVRYSEPPH GLTRTSVTVS DLEPHMNYTF TVEARNGVSG
              430 440 450 460 470 480
LVTSRSFRTA SVSINQTEPP KVRLEGRSTT SLSVSWSIPP PQQSRVWKYE VTYRKKGDSN
              490 500 510 520 530 540
SYNVRRTEGF SVTLDDLAPD TTYLVQVQAL TQEGQGAGSK VHEFQTLSPE GSGNLAVIGG
              550 560 570 580 590 600
VAVGVVLLLV LAGVGFFIHR RRKNQRARQS PEDVYFSKSE QLKPLKTYVD PHTYEDPNQA
              610 620 630 640 650 660
VLKFTTEIHP SCVTRQKVIG AGEFGEVYKG MLKTSSGKKE VPVAIKTLKA GYTEKQRVDF
              670 680 690 700 710 720
LGEAGIMGQF SHHNIIRLEG VISKYKPMMI ITEYMENGAL DKFLREKDGE FSVLQLVGML
              730 740 750 760 770 780
RGIAAGMKYL ANMNYVHRDL AARNILVNSN LVCKVSDFGL SRVLEDDPEA TYTTSGGKIP
              790 800 810 820 830 840
IRWTAPEAIS YRKFTSASDV WSFGIVMWEV MTYGERPYWE LSNHEVMKAI NDGFRLPTPM
              850 860 870 880 890 900
DCPSAIYQLM MQCWQQERAR RPKFADIVSI LDKLIRAPDS LKTLADFDPR VSIRLPSTSG
              910 920 930 940 950 960
SEGVPFRTVS EWLESIKMQQ YTEHFMAAGY TAIEKVVQMT NDDIKRIGVR LPGHQKRIAY
              970
SLLGLKDQVN TVGIPI
```

In other embodiments, the antibody binds to a multiple sites on the portion of the soluble form of EphA2 receptor that corresponds to the extracellular domain of the membrane-bound form of EphA2 receptor. The instant purified antibodies can be produced in accordance with established protocols or using variations thereon.

Also provided are kits for detecting a soluble form of EphA2 receptor in a biological fluid comprising, for use as a detector reagent, a purified antibody that specifically binds to a soluble form of EphA2 receptor, and instructions for use comprising a standard curve for interpolating the quantity of the soluble form of EphA2 receptor in the biological fluid. A standard curve may be prepared in accordance with established protocols or as provided in Example 2, infra, or as a variant thereof. The purified antibody may be directly conjugated to a signal moiety, such as, inter alia, biotin, a fluorophore, a radioactive molecule, a dye. In other embodiments, the kit may further comprise a secondary antibody for binding the purified antibody, wherein the secondary antibody is conjugated to a signal moiety. The purified antibody, and where present, the secondary antibody, may be provided in premeasured aliquots. The instant kits may additionally comprise one or more of a blocking reagent, dilution reagent, buffering reagent, labeling reagent, and wash reagent, any of which may be provided in one or more premeasured aliquots.

### EXAMPLES

### Example 1

### Human Soluble EphA2-R ELISA Assay

An assay was constructed using a goat polyclonal antibody that binds the extracellular domain of the membrane-bound form of the EphA2 receptor (EphA2-R). This reagent was used as a “capture” reagent in the preparation of an ELISA sandwich assay, while the detector reagent was a goat polyclonal antibody labeled with biotin. The streptavidin conjugate was linked to horseradish peroxidase, while the substrate for colorimetric quantitation was TM Blue.

Nunc plates (96 well) were coated with 1 μg/mL anti-human soluble-EphA2-R Goat Polyclonal antibody (R&D Systems, Minneapolis, Minn.; Cat. No. AF3035) and blocked. Human serum and plasma samples were diluted in a sample diluent with goat IgG as a blocker. The standard was a mammalian-expressed form of the human soluble EphA2 (sEphA2) receptor extracellular domain (R&D Systems, Minneapolis, Minn.; Cat. No. 3035-A2). Standards and samples were incubated on the plates for 1 hr, at 37° C. The detector antibody was a biotinylated anti-human soluble-EphA2-R Goat Polyclonal antibody (R&D Systems, Minneapolis, Minn.; Cat. No. BAF3035). The plates were washed six times in phosphate wash and the detector antibody was added to the plates for 1 hr, at 37° C. Plates were washed again six times and a streptavidin-HRP conjugate (Jackson Immunoresearch, West Grove, Pa.) was added to the plates for 1 hr at room temperature. A final wash was performed as before and a TM Blue substrate (Dako, Carpinteria, Calif.) was added to the plates for color development. The reaction was stopped with 2.5N sulfuric acid and plates were read at an absorbance of 450 nm.

### Example 2

### Sample Analysis

A series of human serum and human plasma samples were tested using the prototype ELISA assay described in Example 1 for the presence of circulating EphA2 receptor. Samples were obtained from the Siemens Medical Solutions Diagnostics in-house sample bank, and were originally procured from normal human subjects and subjects in which cancer was known to be present, i.e., by type of cancer, and stage of cancer. A standard curve was constructed using known amounts of recombinant EphA2 extracellular domain protein. The values obtained from the serum and plasma samples were compared to the standard curve in order to interpolate the quantity of circulating EphA2 receptor in the samples. Data are provided in Tables 1-9, below.

TABLE 1
Normal Human Male Serum (NHMS)

| | | Circulating EphA2 Receptor | |
|---|---|---|---|
| Sample ID# | Sample Type | (pg/mL) | Mean |
| 4613 | NHMS | 187.468 | |
| 4614 | NHMS | 278.55 | |
| 4615 | NHMS | 318.828 | |
| 4616 | NHMS | 479.404 | |
| 4617 | NHMS | 242.897 | |
| 4618 | NHMS | 667.632 | |
| 4619 | NHMS | 123.292 | |
| 4620 | NHMS | 432.939 | |
| 4117 | NHMS | 715.469 | |
| 4118 | NHMS | 994.515 | 444.099 |

TABLE 2
Normal Human Female Serum (NHFS)

| | | Circulating EphA2 Receptor | |
|---|---|---|---|
| Sample ID# | Sample Type | (pg/mL) | Mean |
| 4078 | NHFS | 1176.201 | |
| 4079 | NHFS | 1076.918 | |
| 4080 | NHFS | 2079.374 | |
| 4081 | NHFS | 1364.848 | |
| 4082 | NHFS | 2629.435 | |
| 4083 | NHFS | 5578.615 | |
| 4084 | NHFS | 1903.719 | |
| 4085 | NHFS | 1545.998 | |
| 4086 | NHFS | 7615.773 | 2774.542 |

TABLE 3
Normal Human Male Plasma in EDTA

| | | Circulating EphA2 | |
|---|---|---|---|
| Sample ID# | Sample Type | Receptor (pg/mL) | Mean |
| 4517 | Normal Male EDTA | 1217.358 | |
| | Plasma | | |
| 4518 | Normal Male EDTA | 738.471 | |
| | Plasma | | |
| 4519 | Normal Male EDTA | 3591.946 | |
| | Plasma | | |
| 4521 | Normal Male EDTA | 1506.073 | |
| | Plasma | | |
| 4522 | Normal Male EDTA | 505.772 | |
| | Plasma | | |
| 4523 | Normal Male EDTA | 495.964 | |
| | Plasma | | |
| 4524 | Normal Male EDTA | 616.913 | |
| | Plasma | | |
| 4525 | Normal Male EDTA | 477.726 | |
| | Plasma | | |
| 0203-185-00092 | Normal Male EDTA | 624.262 | 1086.054 |
| | Plasma | | |

TABLE 4
Normal Human Female Plasma in EDTA

| | | Circulating EphA2 | |
|---|---|---|---|
| Sample ID# | Sample Type | Receptor (pg/mL) | Mean |
| 4526 | Normal Female EDTA | 2209.868 | |
| | Plasma | | |
| 4529 | Normal Female EDTA | 2574.301 | |
| | Plasma | | |
| 4532 | Normal Female EDTA | 422.621 | |
| | Plasma | | |
| 4534 | Normal Female EDTA | 765.053 | |
| | Plasma | | |
| 4536 | Normal Female EDTA | 499.21 | |
| | Plasma | | |
| 4537 | Normal Female EDTA | 455.461 | |
| | Plasma | | |
| 4539 | Normal Female EDTA | 7820.001 | |
| | Plasma | | |
| 4542 | Normal Female EDTA | 414.541 | |
| | Plasma | | |
| 4509 | Normal Female EDTA | 664.712 | 1758.419 |
| | Plasma | | |

TABLE 5
Lung Cancer Serum

| | | Circulating EphA2 | |
|---|---|---|---|
| Sample ID# | Sample Type | Receptor (pg/mL) | Mean |
| 0010-185-03710 | Lung Cancer Serum | 624.345 | |
| 0010-185-03716 | Lung Cancer Serum | 391.966 | |
| 0011-185-01142 | Lung Cancer Serum | 1982.888 | |
| 0207-185-01281 | Lung Cancer Serum | 1093.11 | |
| 0209-185-00515 | Lung Cancer Serum | 914.988 | |
| 0012-185-01255 | Lung Cancer Serum | 3214.931 | |
| 0207-185-01316 | Lung Cancer Serum | 2476.98 | 1528.458 |

TABLE 6
Kidney Cancer

| | | Circulating EphA2 | |
|---|---|---|---|
| Sample ID# | Sample Type | Receptor (pg/mL) | Mean |
| 0207-185-00093 | Kidney cancer | 988.676 | |
| 0209-185-01367 | Kidney cancer | 2522.629 | |
| 0211-185-00012 | Kidney cancer | 9290.414 | 4267.240 |

TABLE 7
Late Stage Breast Cancer

| | | Circulating EphA2 | |
|---|---|---|---|
| Sample ID# | Sample Type | Receptor (pg/mL) | Mean |
| 0011-185-01146 | Late Stage Breast | 1213.572 | |
| 0101-185-01270 | Late Stage Breast | 656.355 | |
| 0207-185-01321 | Late Stage Breast | 410.745 | 760.224 |

TABLE 8
Prostate Cancer Serum

| | | Circulating EphA2 | |
|---|---|---|---|
| Sample ID# | Sample Type | Receptor (pg/mL) | Mean |
| 3402 S | Prostate Cancer Serum | 280.148 | |
| 3403 S | Prostate Cancer Serum | 941.002 | |
| 3404 S | Prostate Cancer Serum | 544.984 | |
| 3406 S | Prostate Cancer Serum | 186.502 | |
| 3407 S | Prostate Cancer Serum | 2589.159 | 908.359 |

TABLE 9
Late Stage Colon Cancer

| | | Circulating EphA2 | |
|---|---|---|---|
| Sample ID# | Sample Type | Receptor (pg/mL) | Mean |
| 0011-185-01140 | Late Stage Colon | 1317.693 | |
| 0011-185-01141 | Late Stage Colon | 1420.093 | |
| 0012-185-01254 | Late Stage Colon | 1120.826 | |
| 0012-185-01262 | Late Stage Colon | 1220.312 | |
| 0012-185-01275 | Late Stage Colon | 1851.334 | |
| 0101-185-01264 | Late Stage Colon | 1413.959 | |
| 0101-185-01266 | Late Stage Colon | 2682.682 | |
| 0101-185-01267 | Late Stage Colon | 1138.37 | |
| 0101-185-01268 | Late Stage Colon | 956.587 | |
| 0101-185-01271 | Late Stage Colon | 1198.696 | |
| 0101-185-01468 | Late Stage Colon | 9466.375 | |
| 0102-185-00085 | Late Stage Colon | 6118.534 | |
| 0104-185-01092 | Late Stage Colon | 3845.254 | |
| 0106-185-03022 | Late Stage Colon | 7456.842 | |
| 0109-185-00035 | Late Stage Colon | 2159.888 | |
| 0109-185-00036 | Late Stage Colon | 3665.077 | |
| 0109-185-00038 | Late Stage Colon | 1105.207 | |
| 0207-185-01315 | Late Stage Colon | 2893.03 | |
| 0207-185-01317 | Late Stage Colon | 4993.349 | |
| 0207-185-01287 | Late Stage Colon | 7471.417 | |
| 0207-185-01320 | Late Stage Colon | 6943.903 | 3354.258 |

Results demonstrated that the mean value of normal human male plasma (1086.054 pg/mL) and serum (444.099 pg/mL) was lower than that of serum from advanced colon cancer patients (3354.258 pg/mL) and renal cell carcinoma patients (4267.240 pg/mL). In addition, normal human female plasma (1758.419 pg/mL) and serum (2774.542 pg/mL) had higher mean values of soluble EphA2 receptor than normal male serum and plasma. Other cancer serum samples analyzed included lung cancer, prostate cancer, and breast cancer. Results of such analysis indicated that the mean values of prostate cancer serum samples were higher (908.359 pg/mL) than normal male serum samples (444.099 pg/mL), but that mean values of late stage breast cancer serum samples (760.224 pg/mL) were lower than that of normal female serum samples (2774.542 pg/mL).

The reference works, patents, patent applications, and scientific literature that are referred to herein are hereby incorporated by reference in their entirety to the same extent as if each was specifically and individually indicated to be incorporated by reference. Any conflict between any reference cited herein and the specific teachings of this specification shall be resolved in favor of the latter.

What is claimed:

1. A method comprising detecting a soluble form of EphA2 receptor in a biological fluid of a subject.

2. The method according to claim 1 wherein said biological fluid is serum, plasma, sputum, acellular cerebrospinal fluid, acellular saliva, sweat, tears, urine, or any combination thereof.

3. The method according to claim 1 further comprising determining the quantity of said soluble EphA2 receptor, thereby obtaining a sample value.

4. The method according to claim 3 wherein said sample value is determined using an ELISA assay.

5. The method according to claim 3 further comprising comparing said sample value with a reference value.

6. The method according to claim 5, wherein said reference value corresponds to a quantity of soluble EphA2 receptor in a biological fluid of a subject in which a disease state is known to be absent.

7. The method according to claim 6 further comprising assessing the suitability of a therapy regimen based on the comparison between said sample value and said reference value.

8. The method according to claim 7 wherein the therapy regimen is a cellular EphA2 receptor-targeted therapy.

9. The method according to claim 6 further comprising assessing the prognosis with respect to said diseases state for the subject to which the sample value corresponds.

10. The method according to claim 9 wherein said prognosis relates to rate of progression of said disease state, outcome of such disease state, or probability of recurrence of said disease state.

11. The method according to claim 5 wherein said reference value corresponds to the minimum quantity of soluble EphA2 receptor that is necessary to indicate the presence of a disease state.

12. The method according to claim 5, wherein said disease state is cancer.

13. The method according to claim 5, further comprising selecting a therapy regimen based on said comparison of said sample value with said reference value.

14. The method according to claim 3 comprising obtaining a plurality of sample values in a biological fluid of said subject over time.

15. The method according to claim 14, further comprising obtaining an average of said plurality of sample values, and comparing said average with a reference value, wherein said reference value corresponds to the minimum quantity of soluble EphA2 receptor that is necessary to indicate the presence of a disease state.

16. The method according to claim 15, wherein said disease state is cancer.

17. The method according to claim 1, further comprising measuring one or more additional markers in said biological fluid.

18. A method for detecting or aiding the detection of a disease state in a patient comprising determining the quantity of soluble EphA2 receptor in a biological fluid of said patient, wherein an elevated quantity of soluble EphA2 receptor compared to a threshold value indicates the presence of said disease state in said patient.

19. The method according to claim 18 comprising determining a plurality of values corresponding to the quantity of said soluble EphA2 receptor in a biological fluid of said patient over time.

20. The method according to claim 19 further comprising evaluating angiogenic progression over time based on said plurality of values.

21. The method according to claim 18 further comprising selecting a therapy regimen based on said quantity of soluble EphA2 receptor.

22. The method according to claim 18, further comprising measuring one or more additional markers in said biological fluid.

23. A method for monitoring a patient undergoing a therapy regimen comprising:
determining a plurality of values corresponding to the quantity of soluble EphA2 receptor in a biological fluid of said patient over time, wherein an absence of change or a decrease in said values over time indicates a therapeutic effect of said therapy regimen in said patient, and wherein an increase in said values over time indicates the absence of a therapeutic effect of said therapy regimen in said patient.

24. The method according to claim 23 wherein said therapy regimen comprises antiangiogenic treatment.

25. The method according to claim 23 further comprising, based on said plurality of values over time, altering said therapy regimen.

26. The method according to claim 23 further comprising determining a plurality of values corresponding to the quantity of each of one or more additional markers in said biological fluid over time, thereby obtaining a value for each of said one or more additional markers for every timepoint at which a value corresponding to the quantity of soluble EphA2 receptor is determined.

27. A purified antibody that specifically binds to a soluble form of EphA2 receptor.

28. The antibody of claim 27, wherein said antibody is monoclonal

29. The antibody of claim 27, wherein said antibody is polyclonal.

30. A kit for detecting a soluble form of EphA2 receptor in a biological fluid comprising:
an antibody according to claim 27 for use as a detector reagent; and,
instructions for use comprising a standard curve for interpolating the quantity of said soluble form of EphA2 receptor in said biological fluid.

31. The kit according to claim 30 wherein said antibody is conjugated to a signal moiety.

32. The kit according to claim 31, wherein said signal moiety comprises biotin, a fluorescent moiety, a radiological moiety, or a dye.

33. The kit according to claim 30 further comprising a secondary antibody for binding said antibody that binds to a soluble form of EphA2 receptor, wherein said secondary antibody is conjugated to a signal moiety.

34. A method for monitoring a patient in which a disease state is known to be present comprising determining the quantity of soluble EphA2 receptor in a biological fluid of said patient, thereby obtaining a sample value, and comparing said sample value with a reference value.

35. The method according to claim 34 wherein said disease state is cancer.

36. The method according to claim 34 wherein said reference value corresponds to the quantity of soluble EphA2 receptor known to be present in a biological fluid of a patient at a known stage of said disease state.

37. The method according to claim 34 wherein said reference value corresponds to the quantity of soluble EphA2 receptor known to be present in a biological fluid of said patient at an earlier time point.

38. The method according to claim 34 further comprising determining a plurality of values corresponding to the quantity of soluble EphA2 receptor in a biological fluid of said patient over time.

39. The method according to claim 38 further comprising conducting a comparison among said plurality of values, thereby determining the absence or presence of one or more trends in the quantity of said soluble EphA2 receptor in said biological fluid of said patient over time.

40. The method according to claim 38 further comprising conducting a comparison between said plurality of values and a plurality of reference values, wherein each of said plurality of reference values corresponds to the quantity of soluble EphA2 receptor known to be present in a biological fluid of a patient at a known stage of said disease state, and wherein each of said plurality of reference values corresponds to a different stage of said disease state.

41. The method according to claim 34, further comprising measuring one or more additional markers in said biological fluid.


SEQUENCE LISTING (1 sequence)

SEQ ID NO: 1 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERNNFELNFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI